(19) United States
(12) Patent Application Publication
(10) Pub. No.: US 2010/0304470 A1
(43) Pub. Date: Dec. 2, 2010

(54) MICROFLUIDIC CHIP

(75) Inventors: YA-FU CHUANG, Tainan County (TW); I-EN LIN, Miaoli County (TW)

Correspondence Address: WPAT, PC;INTELLECTUAL PROPERTY ATTORNEYS, 7225 BEVERLY ST., ANNANDALE, VA, 22003, US

(73) Assignee: INDUSTRIAL TECHNOLOGY RESEARCH INSTITUTE, HSIN-CHU (TW)

(21) Appl. No.: 12/497,781

(22) Filed: Jul. 6, 2009

(30) Foreign Application Priority Data
May 27, 2009 (TW) 098117679

(51) Int. Cl. C12M 1/34

(52) U.S. Cl. 435/287.2

(57) ABSTRACT

A microfluidic chip including a substrate and at least a channel set is provided. The substrate has a surface, and the channel set is formed in the substrate and includes a channel, at a least a filler fillister, and at least a well fillister. The filler fillister and the well fillister are all connected to the channel, and the channel passes through the well fillister. The depth of the well fillister relative to the surface is longer than the depth of the channel relative to the surface. Hence, the well fillister can hinder the fluid flow in the channel and be used as a valve.

### FIELD OF THE INVENTION

The present invention relates to a tool for biological sample assay, and more particularly, to a microfluidic chip adapted for biological applications.

### BACKGROUND OF THE INVENTION

Enzyme-linked immunosorbent assay, also called ELISA, is a biochemical technique used mainly in immunology to detect the presence of an antibody or an antigen in a sample. In simple terms, in ELISA an unknown amount of antigen is affixed to a surface, and then a specific antibody is washed over the surface so that it can bind to the antigen. This antibody is linked to an enzyme, and in the final step a substance is added that the enzyme can convert to some detectable signal. Thus in the case of fluorescence ELISA, when light of the appropriate wavelength is shone upon the sample, any antigen/antibody complexes will fluoresce so that the amount of antigen in the sample can be inferred through the magnitude of the fluorescence.

Performing a conventional ELISA usually involves a great deal of manual labor for executeing those many complex experimental procedures in a step-by-step manner. Thus, it might take several hours or even more than a day just to get any result from a conventional ELISA.

### SUMMARY OF THE INVENTION

The object of the present invention is to provide a microfluidic chip adapted for ELISA.

To achieve the above object, the present invention provides a microfluidic chip, comprising: a substrate, having a surface; and at least a channel set, each being formed in the substrate and configured with a channel, at least a filler fillister in fluid communication with the channel, and at least a well fillister in fluid communication with the channel; wherein each filler fillister and well fillister of the same channel set are all connected to its channel as the channel is arranged passing through each well fillister; and the depth of the well fillister relative to the surface of the substrate is larger than the depth of its corresponding channel relative to the surface.

Accordingly, each well fillister is able to hinder the flowing of a fluid inside its corresponding channel and thus be used as a well-type valve for controlling the flowing of the fluid. Thus, the microfluidic chip of the invention not only is adapted for ELISA, but also can be adapted for other biological or chemical applications.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention and wherein:

FIG. 1A is a top view of a microfluidic chip according to a first embodiment of the invention.

FIG. 1B is an enlarged diagram showing a portion of FIG. 1A.

FIG. 1C is an I-I sectional view of FIG. 1B.

FIG. 2A to FIG. 2C are schematic diagrams showing how a fluid is flowing in a channel of FIG. 1C.

FIG. 3A is a top view of a microfluidic chip according to a second embodiment of the invention.

FIG. 3B is an enlarged diagram showing a portion of FIG. 3A.

FIG. 3C is a J-J sectional view of FIG. 3B.

### DESCRIPTION OF THE EXEMPLARY EMBODIMENTS

For your esteemed members of reviewing committee to further understand and recognize the fulfilled functions and structural characteristics of the invention, several exemplary embodiments cooperating with detailed description are presented as the follows.

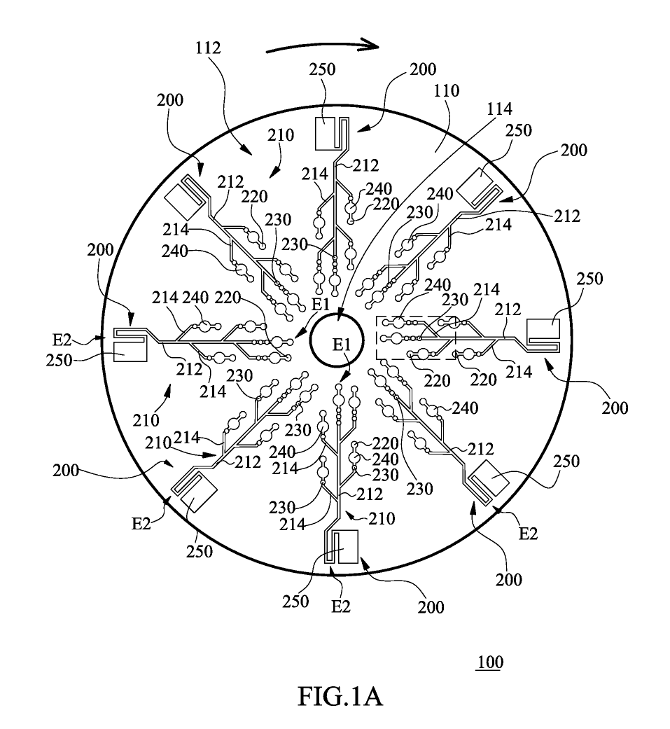

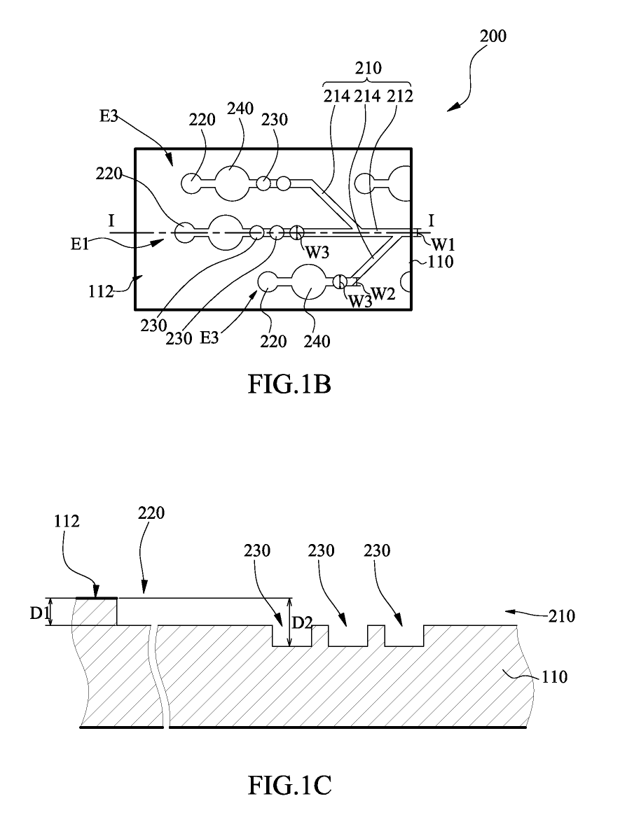

FIG. 1A is a top view of a microfluidic chip according to a first embodiment of the invention; and FIG. 1B is an enlarged diagram showing an area defined in the microfluidic chip of FIG. 1A enclosed by a dotted frame. In FIG. 1A and FIG. 1B, the microfluidic chip 100 is composed of a substrate 110 and a plurality of channel sets 200, in which the plural channel sets 200 are formed in the substrate 110. It is noted that the amount of channel sets 200 being formed in the substrate 110 is not limited by the present embodiment, and thus it is possible to have only one channel set 200 in the microfluidic chip 100.

As shown in FIG. 1A, the substrate 110 is formed with a surface 112 and a via hole 114 while allowing the plural channel sets 200 to be exposed on the surface 112. In this embodiment, the substrate 110 is substantially a disc while enabling the surface 112 to be a round-shaped surface. Thereby, the plural channel sets 200 are equiangularly arranged around the circumference of the surface 112. In addition, the via hole 114 is formed on the surface 112 and extending along the axis of the disc-shape substrate 110, by that the via hole 114 is substantially located at the center of the surface 112.

In this first embodiment, each channel set 200 includes a channel 210, a plurality of filler fillisters 220, a plurality of well fillisters 230 and a plurality of storage fillisters 240. However, it is not limited thereby since it is possible to have only one filler fillister 220, one well fillister 230 and no storage fillister 240 in each channel set 200 according to other embodiments of the invention. That is, each channel set 200 can be formed with only one filler fillister 220 and one well fillister 230. It is noted that the amounts of filler fillisters 220, well fillisters 230 and storage fillisters 240 in each channel set 200 shown in the first embodiment of FIG. 1A are only for illustration, and thus are not limited thereby.

In FIG. 1A, all the filler fillisters 220, well fillisters 230 and storage fillisters 240 are formed in fluid communication with their corresponding channel 210; and each storage fillister 240 is formed at a position between its corresponding filler fillister 220 and well fillister 230 as the channel 210 is arranged passing through the storage fillister 240 and the well fillister 230.

In detail, each channel 210 is composed of a main duct 212 and a plurality of manifolds 214, in which each manifold 214 is formed in fluid communication with the main duct 212 while arranging all the corresponding filler fillisters 220, well fillister 230 and storage fillisters 240 of the same channel set 200 to be in fluid communication with the main duct 212 and the plural manifolds 214. In addition, the main duct 212 and the manifolds 214 are arranged for allowing each to pass through a number of the well fillisters 230 and at least one storage fillister 240.

Accordingly, the main duct and each of the manifolds 214 are respectively configured with a starting end E1 and a terminating end E2 corresponding to the starting end; and each main duct 212 is arranged to be in fluid communication with more than one manifolds 214. It is noted that all the starting ends E1 are located at positions closer to the via hole 114 than their corresponding terminating ends E2. Moreover, each channel set 200 can further comprises a gathering fillister 250 which is formed in fluid communication with the corresponding main duct 212. Thereby, each channel set 200 is arranged for allowing its corresponding filler fillisters 220 to be located at the starting ends E1 and its corresponding gathering fillisters 250 to be positioned at the terminating ends E2.

In this embodiment, the main duct 212 and the plural manifolds 214 are formed in fluid communication with the filler fillisters 220, the well fillisters 240 and storage fillisters 230, and the filler fillisters 220 are designed to be filled with different samples or reaction fluids according to actual requirement. Thereby, when the substrate 110 is enabled to rotate, the samples or reaction fluids in the filler fillisters 220 will flow into their corresponding storage fillisters 240 and then into the well fillisters 230 in respective. As soon as the samples or reaction fluids overflowed their corresponding well fillisters 230, they will continuous their journey and flow into their corresponding main ducts 212 where there are mixed.

Please refer to FIG. 1C, which is an I-I sectional view of FIG. 1B. As shown in FIG. 1B and FIG. 1C, for each channel set 200, the depth D2 of each well fillister 230 relative to the surface 112 of the substrate 110 is larger than the depth D1 of its corresponding channel 210 relative to the surface 112. In detail, assuming the depths of the main duct 212 and manifolds 214 of one channel set 200 are both equal to D1 and the depth of the well fillisters 230 of the same channel set 200 is equal to D2, the depth D1 is smaller than D2.

Moreover, for the same channel set 200, the depths of the fillisters 220 and storage fillisters 240 relative to the surface 112 of the substrate 110 can also be equal to D1, i.e. for the same channel set 200, the bottoms of the filler fillisters 220, storage fillisters 240, main duct 212, manifolds 214 are substantially located on the same level. However, they are not limited thereby that it is possible to have a channel set 200 whose storage fillisters 240 are formed with a depth larger than D1, or even equal to D2.

Moreover, for the same channel set 200 in this embodiment, the width W3 of the well fillisters 230 can be formed larger than the width W1 of the main duct 212, or the width W2 of the manifolds 214. It is noted that he width W1 of the main duct 212 can equal to the width W2 of the manifolds 214. Nevertheless, they are not limited thereby that it is possible to have a channel set 200 whose width W3 of the well fillisters 230 can be formed equal to the width W1 of the main duct 212, or the width W2 of the manifolds 214.

The substrate 110 can be made of a plastic, such as polymethylmethacrylate (PMMA), or a glass, so that it is made to be a transparent component for allowing the same to be transmitted by a light of at least one wavelength. Thus, the substrate 110 can be a transparent, colorless component, or can be a filter for converting a white light into red, blue, green or yellow lights, and so on.

When the substrate 110 is made of a plastic, it can be made of any common plastic substrates used for making ordinary compact disc (CD). That is, the surface 112 of the substrate 110 is formed with a size equal to the surface size of any common CD. In addition, the channel set is fabricated by the use of a laser beam; and the laser beam can be emitted from a laser device, such as femtosecond laser equipment or a picosecond laser equipment. Notably that the picosecond laser equipment is substantially an excimer laser device.

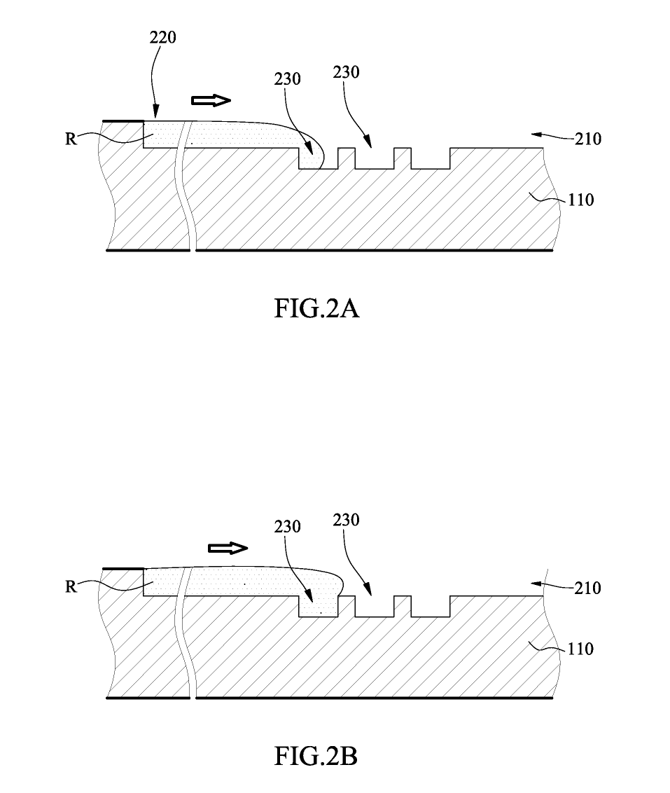

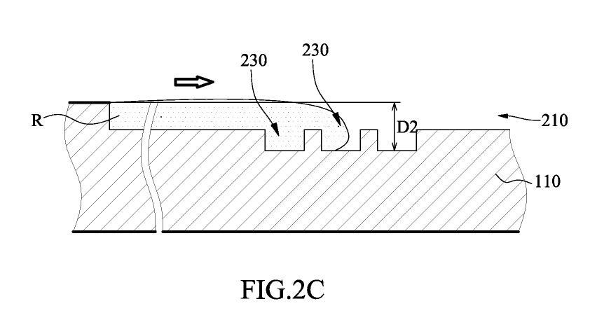

Please refer to FIG. 2A to FIG. 2C, which are schematic diagrams showing how a fluid is flowing in a channel of FIG. 1C. In FIG. 2A, When a substrate 110 whose filler fillisters 220 are filled with reaction fluids R such as antigens, antibodies or enzymes is being enabled to rotate using its via hole 114 as rotation axis, the centrifugal force caused by the rotation will force the reaction fluid R to flow in a direction away from the via hole 114.

As shown in FIG. 2A, as soon as the reaction fluid R flowing in a channel 210 reaches a well fillister 230, the flow of the reaction fluid R will be hold until the well fillister 230 is filled. As shown in FIG. 2B, after the well fillister 230 is filled by the reaction fluid R, the surface tension force at the liquid-air interface of the reaction fluid R is going to work against the centrifugal force caused by the rotation of the substrate 110. Thus, if the rotation speed of the substrate 110 is maintained unchanged without increasing, the reaction fluid R will be hold inside the well fillister 230 without overflowing and thus stopping from flowing.

As shown in FIG. 2C, when the rotation speed of the substrate 110 is increased for generating a centrifugal force larger than the surface tension force, the reaction fluid R will be pressed to overflow the well fillister 230 so that the reaction fluid R can flow again in the channel 210 passing the well fillister 230 and possibly into another well fillister 230. Therefore, by the balance between the surface tension force of the reaction fluid R and the centrifugal force caused from the rotation of the substrate 110, each well fillisters 230 can work as a valve for controlling the flow of the reaction fluid R.

Moreover, the well fillisters 230 is this embodiment can be formed of the same depth or different depths. For instance, it is possible to have one of the well fillister 230 to be formed with a depth different from those of the other well fillister 230 in the same channel set 200. Moreover, since each channel set 200 is formed by the use of a laser beam, the surface roughness of the well fillister 230 can be controlled by the laser energy, scanning frequency, polarization, and overlay relating to the laser beam. In addition, after forming the well fillister 230 by laser, a chemical micro-etching process can be performed for further controlling the surface roughness of the well fillisters 230.

It is noted that the rougher the surface of a well fillister is including its bottom and sidewall, the harder the reaction fluid is able to flow; and vice versa. Thus, by designing the well fillister 230 with a surface roughness, the rotation speed of the substrate 110 is determined that is the speed capable of enabling the reaction fluid R to overflow the well fillister 230 and continue flow.

From the foregoing description, it is noted that by designing well fillisters 230 of different sizes and surface roughness in those manifolds 214 that are formed in fluid communication with a same main duct 212, different reaction fluids R flowing in those different manifolds are controlled and enabled to overcome their respective surface tension force under different rotation speeds of the substrate 110 so that the mixing time and sequence relating to those different reaction fluids R in the same main duct 212 can be controlled. Thereby, the microfluidic chip of the invention can be adapted for various biological application or chemical experiments.

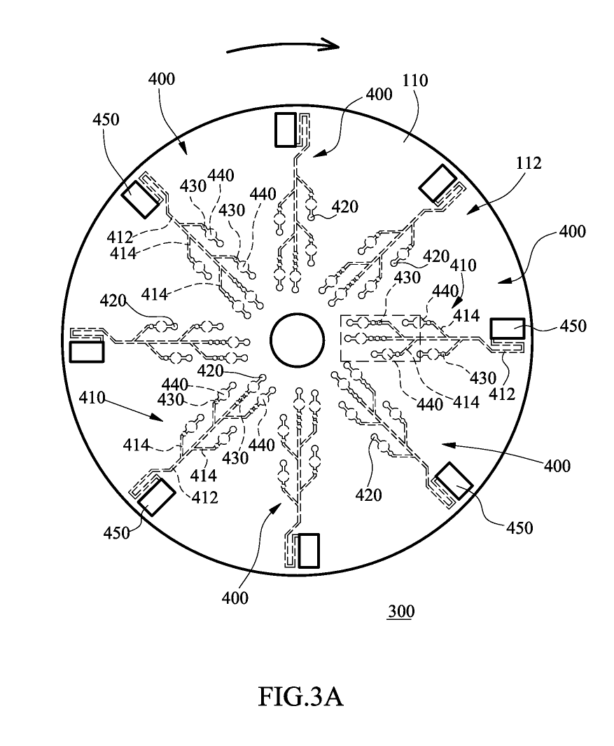

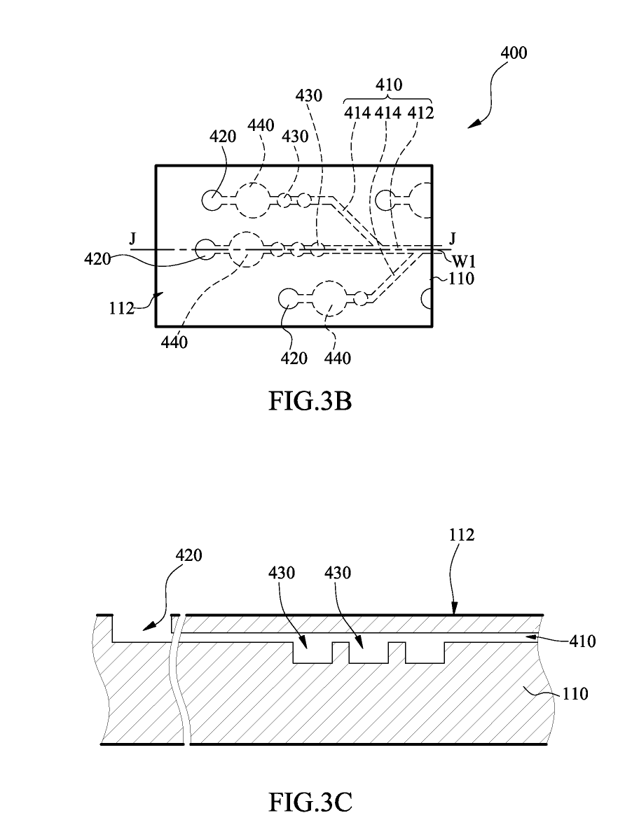

FIG. 3A is a top view of a microfluidic chip according to a second embodiment of the invention, FIG. 3B is an enlarged diagram showing an area defined in the microfluidic chip of FIG. 3A enclosed by a dotted frame, and FIG. 3C is a J-J sectional view of FIG. 3B. The microfluidic chip 300 shown in the second embodiment is similar to the microfluidic chip 100 of the first embodiment in function and structure, but is different in that: all the channel 410, the well fillister 430 and storage fillister 440 are embedded inside the substrate 110.

As shown in FIG. 3A, the microfluidic chip 300 includes a substrate 110 and a plurality of channel sets 400 formed inside the substrate 110, in which each channel set 400 is composed of a channel 410, a plurality filler fillisters 420, a plurality of well fillisters 430, a plurality of storage fillisters 440 and a plurality of gathering fillister 450. Moreover, the channel sets 400 are equiangularly arranged around the circumference of the surface 112 of the substrate 110.

Similarly, each channel 410 is composed of a main duct 412 and a plurality of manifolds 414, in which each manifold 414 is formed in fluid communication with the main duct 412. As shown in FIG. 3B, other than portions of the filler fillisters 42 and gathering fillisters 450 are exposed on the surface 112 of the substrate 110, the main duct 412, the manifolds 414, the well fillisters 430 and the storage fillisters 440 are all being formed buries inside the substrate. It is noted that there can be holes being formed respectively at positions on top of each filler fillister 420 and each gathering fillister 450 for enabling those fillisters to communicate with outside air therethrough while preventing the reaction fluids received therein from spraying out on the surface 112.

Accordingly, the channels 410 can be referred as tunnels buries under the surface 112 of the substrate 110. Therefore, when the reaction fluids are driven to flow inside the channels 410, they can be prevented from spraying out on the surface 112 and thus enable any biological testing or chemical experiment using those reaction fluids to carry on smoothly.

The substrate 110 is made of a plastic or glass, that it is transparent component for allowing the same to be transmitted by a light. Accordingly, it is possible to use laser for forming the channel sets 400 inside the substrate 110. In an exemplary embodiment of the invention, a picosecond laser equipment, such as an excimer laser device, can be used for emitting a laser beam to heat up the internal of the substrate 110 in a manner that a specific internal portion of the substrate 110 is melted for forming the channel sets 400.

Except for the picosecond leaser equipment, it is able to use femtosecond laser equipment to form the channel sets 400 inside the substrate 110 which is different from the picosecond laser that the laser beam from a femtosecond laser equipment can break the binding between molecules directly for enabling an ablation effect inside the substrate 110 without causing a lot of heat being generated inside the substrate 110. Thus, by using laser beam emitted from a femtosecond laser equipment to form the channel sets 400, the situation of the substrate 110 being deteriorated by high temperature can be prevented.

Moreover, except for the forming of channel set 400 inside the substrate 110, the laser beam emitted from a femtosecond laser equipment can also be used in a cleaning procedure for removing the debris from the laser ablation out of the substrate 110 so as to prevent the channel sets 400 from being clogged by such debris and thus enabling the reaction fluids to flow therein without obstruction.

For clarity, all those main ducts 412, manifolds 414, filler fillisters 420, well fillisters 430, storage fillisters 440 and gathering fillisters 450 are shaped and arranged similar to those described in the first embodiment, and thus are not described further herein.

In addition, it is possible to have only one channel set 400 being formed in the microfluid chip 300 of the invention; and the channel set 400 can be configured with only one filler fillister 420, one well fillister 430 and no storage fillister 440 in each channel set 200 according to other embodiments of the invention. That is, each channel set 400 can be formed with only one filler fillister 420 and one well fillister 430. It is noted that the amounts of filler fillisters 420, well fillisters 430 and storage fillisters 440 in each channel set 400 shown in the embodiment of FIG. 3A and FIG. 3B are only for illustration, and thus are not limited thereby.

To sum up, by designing well fillisters with depths larger than their corresponding channels, the microfluidic chip is able to work as a valve for controlling the flow of the reaction fluids by the balance between the surface tension force of the reaction fluid and the centrifugal force caused from the rotation of the substrate. Therefore, the microfluidic chip adapted for ELISA or other biological applications as well as chemical applications.

Notably, as the channels and well fillisters can be buried inside the substrate, the reaction fluids flowing in the channels can be prevented from spraying out on the surface of the substrate during the rotation of the substrate. Thereby, the microfluidic chip can prevent the reaction fluids flowing in different channel sets from mixing with each other before they are intended to so that it is able to test as many samples as possible at the same time using the same microfluidic chip without worrying the interference between different tests.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

What is claimed is:

1. A microfluidic chip, comprising:
a substrate, having a surface; and
at least one channel set, each being formed in the substrate and configured with a channel, at least one filler fillister in fluid communication with the channel, and at least one well fillister in fluid communication with the channel;
wherein, each filler fillister and well fillister of the same channel set are all connected to its channel as the channel is arranged passing through each well fillister; and the depth of the well fillister relative to the surface of the substrate is larger than the depth of its corresponding channel relative to the surface.

2. The microfluidic chip of claim 1, wherein the substrate is substantially a disc while enabling the surface to be a round-shaped surface.

3. The microfluidic chip of claim 2, wherein when there are a plurality of said channel sets being formed in the microfluidic chip, the plural channel sets are equiangularly arranged around the circumference of the surface.

4. The microfluidic chip of claim 2, wherein the substrate further comprises a via hole, being formed on the surface at the center thereof.

5. The microfluidic chip of claim 1, wherein the at least one channel set further comprises: at least one storage fillister, each formed in fluid communication with the corresponding channel at a position between its corresponding filler fillister and well fillister as the channel is arranged passing through each storage fillister.

6. The microfluidic chip of claim 1, wherein the channel includes a main duct in fluid communication with the at least one filler fillister and well fillister of the same channel set while arranging the main duct to pass each well fillister; and the main duct, being configured with a starting end and a terminating end corresponding to the starting end, is positioned for enabling its corresponding filler fillister to be located at the starting end.

7. The microfluidic chip of claim 6, wherein the starting end is located at a position closer to the via hole than the terminating end.

8. The microfluidic chip of claim 6, wherein each channel set further comprises a gathering fillister formed in fluid communication with the main duct while being located at the terminating end thereof.

9. The microfluidic chip of claim 6, wherein when there are a plurality of said filler fillisters and well fillisters configured in one corresponding channel set, the channel of the channel set is formed with a plurality of manifolds in fluid communication with its main duct in a manner that the plural fill fillisters and well fillisters are connected to the main duct and the plural manifolds while allowing each of the plural manifolds to pass one well fillister selected from the plural well fillisters.

10. The microfluidic chip of claim 9, wherein the channel set further comprises a plurality of storage fillisters formed in fluid communication with its main duct and manifolds as the main duct and the manifolds are arranged passing the storage fillisters; and each storage fillister is located at a position between one filler fillister selected from the plural filler fillisters and one well fillister neighboring to the selected filler fillister.

11. The microfluidic chip of claim 9, wherein each of the plural manifolds is extending in a direction from its corresponding main duct to the via hole.

12. The microfluidic chip of claim 9, wherein reaction fluids respectively flowing in the plural manifolds are enabled to flow from the filler fillisters corresponding to the plural manifolds through the corresponding storage fillisters and well fillisters into the main duct where they are mixed.

13. The microfluidic chip of claim 1, wherein the at least one channel set is formed exposing on the surface.

14. The microfluidic chip of claim 1, wherein the channel and the well fillister as well as at least a storage fillister in fluid communication with the channel are embedded inside the substrate while allowing the filler fillister and at least a gathering fillister to be exposed on the surface of the substrate.

15. The microfluidic chip of claim 1, wherein the substrate is made of a material selected from the group comprising of: a plastic or a glass.

16. The microfluidic chip of claim 1, wherein the substrate is transparent component for allowing the same to be transmitted by a light.

17. The microfluidic chip of claim 1, wherein the channel set is fabricated by the use of a laser beam.

18. The microfluidic chip of claim 17, wherein the laser beam is emitted from a femtosecond laser device.

19. The microfluidic chip of claim 17, wherein the laser beam is emitted from a excimer laser device.

20. The microfluidic chip of claim 14, wherein there are holes formed respectively at positions on top of each filler fillister and each gathering fillister for enabling those fillisters to communicate with outside air therethrough.
